(19) United States
(12) Patent Application Publication
(10) Pub. No.: US 2025/0018186 A1
(43) Pub. Date: Jan. 16, 2025

(54) MENSTRUAL PAIN MANAGEMENT DEVICE

(71) Applicant: JUNO TECHNOLOGIES INC, MONTREAL (CA)

(72) Inventors: Nanette SENE, SAINT-LAURENT (CA); Lynn DOUGHANE, SAINT-LAURENT (CA)

(73) Assignee: JUNO TECHNOLOGIES INC, MONTREAL (CA)

(21) Appl. No.: 18/764,497

(22) Filed: Jul. 5, 2024

Related U.S. Application Data
(60) Provisional application No. 63/513,057, filed on Jul. 11, 2023.

(51) Int. Cl. A61N 1/36; A61F 7/00; A61N 1/04; A61N 1/08; H02J 50/05; H02J 50/10

(52) U.S. Cl. CPC A61N 1/36021; A61N 1/0456; A61N 1/0492; A61N 1/08; A61F 7/00; H02J 50/05; H02J 50/10

(57) ABSTRACT

The present menstrual pain management device comprises a base layer, an electrode layer, a heating layer, and an electronic layer. The base layer is adapted for being removable affixed to skin. The electrode layer covers at least two areas longitudinally located along the base layer. The electronic layer comprising a control module in electric connection with the electrode layer and the heating layer. The control module controls operation of the electrode layer and the heating layer. The electronic layer covers the electrode layer and the heating layer.

### TECHNICAL FIELD

The present disclosure relates to the field of menstrual pain management and more particularly to menstrual pain management devices.

### BACKGROUND

Menstrual pain is a condition that accompanies or precedes menses. The intensity of menstrual pain varies from one person to another, and for different reasons: period of the month, flow of menses, hormone levels, stress level, time of day, etc.

Menstrual pain may adversely affect daily activities and render the person affected incapable of pursuing regular daily activities. This condition may be experienced occasionally or systematically every month for up to several days.

Menstrual pain management options typically include the following types: pharmaceutical or non-pharmaceutical. Not all menstrual pain sufferers react favorably to the pharmaceutical options. Non-pharmaceutical options often include applying warmth on the abdomen or using a tens machine. Transcutaneous Electrical Nerve Simulation (TENS) machine do bring some level of relief when the tens are properly positioned on the abdomen. However, TENS machines include wires for interconnecting the electrode to a control module, and the control module must be carried in a pocket. This renders the use of TENS machines suitable for home use, but not as convenient for use during regular activities outside of the home.

There is therefore a need for a new menstrual pain management device which is simple to use, portable and operated discretely.

### SUMMARY

According to a first aspect, the present disclosure relates to a menstrual pain management device which comprises a base layer, an electrode layer, a heating layer and an electronic layer. The base layer is adapted for removable adhesion to skin. The electrode layer coveres at least two areas longitudinally located along the base layer. The electronic layer comprises a control module in electric connection with the electrode layer and the heating layer. The control module controls operation of the electrode layer and the heating layer. The electronic layer covers the electrode layer and the heating layer.

According to a particular aspect, the electrode layer and the heating layer are printed and superposed.

According to another particular aspect, the control module further comprises manual controls for controlling operation of the electrode layer and the heating layer, and the electronic layer further provides access to the manual controls of the control module.

According to another particular aspect, the user controls include an intensity level for the heating layer.

According to yet another particular aspect, the user controls include at least one of the following: an intensity level for the electrode layer, a mode selection for selecting an electric wave pattern for the electrode layer and a frequency of the electric wave pattern.

According to another particular aspect the base layer is shaped as a trapezoid.

According to another particular aspect, the control module further comprises a rechargeable battery for powering the electronic module, the electrode layer and the heating layer, and an input connector for receiving a connector of an electric cable for recharging the rechargeable battery.

According to yet another particular aspect, a periphery of the electronic layer is fused to the base layer to define a water-resistant shell around the electrode layer, the heating layer and the control module.

According to another particular aspect, the electronic layer covers the controls of the control module.

According to yet another particular aspect, the electrode layer defines a central aperture.

According to another particular aspect, the control module further comprises a wireless communication module for communicating with a wireless electronic device.

According to yet another particular aspect, the control module wirelessly communicates a recharge level of the rechargeable battery to the wireless electronic device.

According to another particular aspect, the control module is adapted for controlling the electrode layer to generate a haptic signal indicative of a low recharge level of the rechargeable battery.

According to yet another particular aspect, the control module includes a mode button for concurrently controlling the electrode layer and heating layer.

According to another particular aspect, the mode button provides a low control for concurrently controlling the electrode layer on low intensity and the heating layer on low intensity.

According to yet another particular aspect, the mode button provides a high control for concurrently controlling the electrode layer on high intensity and the heating layer on high intensity.

According to another particular aspect, the base layer and the electronic layer are made of a medical grade silicon.

According to yet another particular aspect, the electrode layer comprises a similar design of electric wire in each one of the at least two areas longitudinally located along the base layer.

According to another particular aspect, the heating layer defines a belt along the base layer.

According to yet another particular aspect, the electronic layer further comprises a rechargeable battery for powering the electronic module and a wireless charging module for wirelessly recharging the rechargeable battery.

According to another particular aspect, the wireless charging module is one of an inductive wireless charging module and a capacitive wireless charging module.

According to yet another particular aspect, the control module further comprises a feedback button for receiving feedback from a user of the menstrual pain management device, the control module adapting the control of the electrode layer and heating layer in response to the feedback received from the user.

### BRIEF DESCRIPTION OF THE DRAWINGS

Embodiments of the disclosure will be described by way of example only with reference to the accompanying drawings, in which:

FIG. 1 is a perspective top side view of an example of the present menstrual pain management device;

FIG. 2 is a side view of the menstrual pain management device of FIG. 1;

FIG. 3 is a front view of the menstrual pain management device of FIG. 1;

FIG. 4 is a top view of an intermediate layer;

FIG. 5 is an exploded view of the menstrual pain management device of FIG. 1; and

FIG. 6 is a schematic functional diagram representing the components of the control module 145.

### DETAILED DESCRIPTION

The foregoing and other features will become more apparent upon reading of the following non-restrictive description of illustrative embodiments thereof, given by way of example only with reference to the accompanying drawings. Like numerals represent like features on the various drawings.

Various aspects of the present disclosure generally address problems with current menstrual pain management devices, including the difficulty of properly positioning TENS, the wires connecting the TENS to the control device, and the difficulty to use such TENS machine while out of the house.

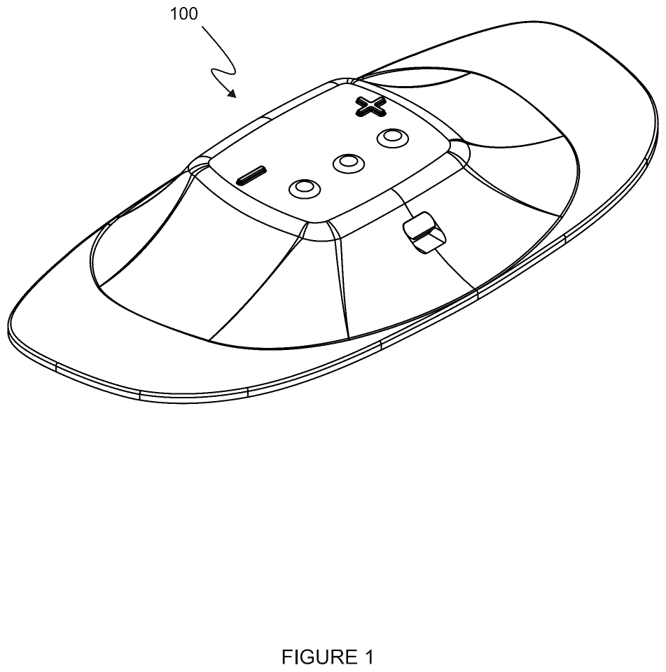

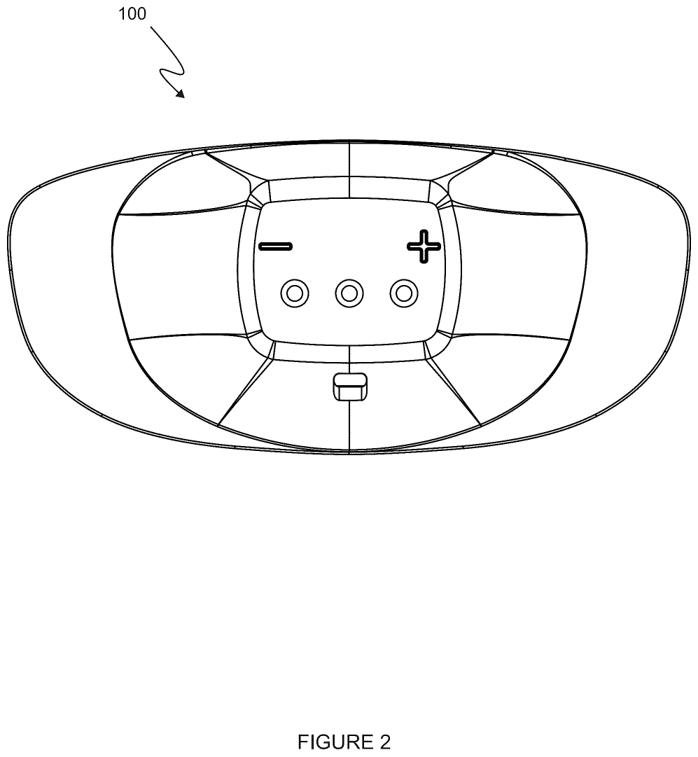

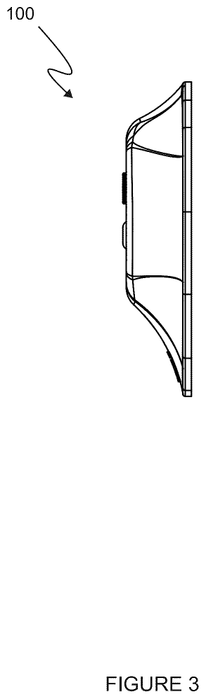

Referring concurrently to FIGS. 1-5 there are shown various views of an example of the present menstrual pain management device. The example shown on FIGS. 1-5 is for explaining the concepts, construction and operation of the present menstrual pain management device and should not be interpreted to limit the present menstrual pain management device to the design and implementation illustrated therein.

The menstrual pain management device 100 is composed of four layers: a base layer 110, an electrode layer 120, a heating layer 130 and an electronic layer 140. The four-layer configuration provides a menstrual pain management device 100 that is simple to operate, which can be discretely used and very portable.

The base layer 110 receives the other three layers: the electrode layer 120, the heating layer 130 and the electronic layer 140. The base layer 110 is made of flexible material. The base layer 110 is adapted to removably adhere to skin. For example, the base layer 110 may be adapted to receive a removable skin adhesive which can be changed after use. Alternatively, the base layer 110 may receive a washable adhesive liquid for removably affix the base layer onto skin. In yet another alternative, the material of the base layer 110 may become adhesive when humid. For example, the base layer 110 may be made of medical-grade silicone.

The base layer 110 is shaped and sized to cover the area of the abdomen of a user impacted by menstrual pain. For example, the base layer 110 may be shaped as a rectangle, a trapezoid, an oval, a butterfly, or any other shape which is adapted for receiving the other three layers of the present menstrual pain management device 100 while properly positioning the electrode layer 120 and the heating layer 130 over the area of the abdomen of the user impacted by menstrual pain.

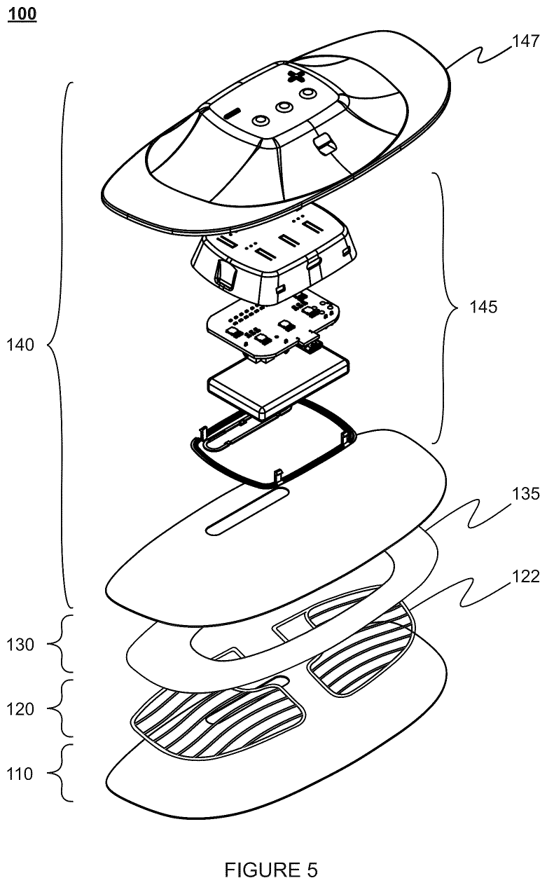

The electrode layer 120 sits over the base layer 110. The electrode layer 120 defines two electrodes 122. The two electrodes 122 are positioned between a center of the base layer 110 and opposite sides thereof. The electrode layer 120 covers at least two areas longitudinally located along the base layer 110. The electrode layer 120 comprises two electrodes 122 created by electric wire shaped in similar design on each side of the electrode layer 120. The design of the electric wire of each electrode 122 may comprise multiple interconnected electric wires electrically connected in serial, in parallel, or in a combination of serial and parallel electrical connections. The electric wire of each electrode 122 is selected to be capable of providing Transcutaneous Electrical Nerve Simulation (TENS) when the menstrual pain management device 100 is installed on a user's abdomen and the electronic layer 140 provides an electric current to the electrodes 122. The design of electric wire shown on FIG. 5 is for example only.

To minimize electrical consumption while optimizing the area over which TENS is provided, the electrodes 122 may be shaped to be positioned towards the sides of the menstrual pain management device 100 while not extending over a center portion thereof, so as to define a central aperture between the electrodes 122. Furthermore, the gauge of the electric wire and the design of the electrodes 122 are selected to keep electric consumption to a minimum while optimizing the TENS when used. The electrode layer 120 is flexible to adapt to the shape of the abdomen of the user. The terminology electrode layer refers to both implementations where the electrodes 122 are designed as a layer inserted between the base layer 110 and the electronic layer 140, or as two separate electrodes 122 affixed therebetween.

The heating layer 130 is also designed to minimize electrical consumption while optimizing the area over which the heating layer provides warmth. For doing so, the heating layer 130 may be shaped as a belt adjacent a periphery of the base layer 110. Alternatively, the heating layer 130 may be shaped as another 2D geometric shape over a central area of the base layer 110. The heating layer 130 is adapted to be inserted between the base layer 110 and the electronic layer 140, below or above the electrode layer 120. The heating layer 130 is also flexible to follow a shape of the abdomen of a user. The heating layer 130 is composed of a heating element 135 and a structure for affixing the heating element between the base layer 110 and the electronic layer 140. The heating layer 130 and the electrode layer 120 may be printed, and more particularly may be printed as superposed on one another. Although the present specification describes the electrode layer 120 and the heating layer 130 as separate layers, such interpretation is limited to the functional aspects of the electrode layer 120 and the heating layer 130.

The electronic layer 140 comprises a control module 145 in electric connection with the electrode layer 120 and the heating layer 130. The control module 145 controls operation of the electrodes 122 of the electrode layer 120 and the heating element 135 of the heating layer 130. The electronic layer 140 further comprises a cover 147 which covers the control module 145, the electrode layer 120 and the heating layer 130. The control module 145 electrically connects with the electrodes 122 of the electrode layer 120 and with the heating element 135 of the heating layer 130 with any means known in the art to electrically interconnect components, such as through wires, traces, connectors, etc.

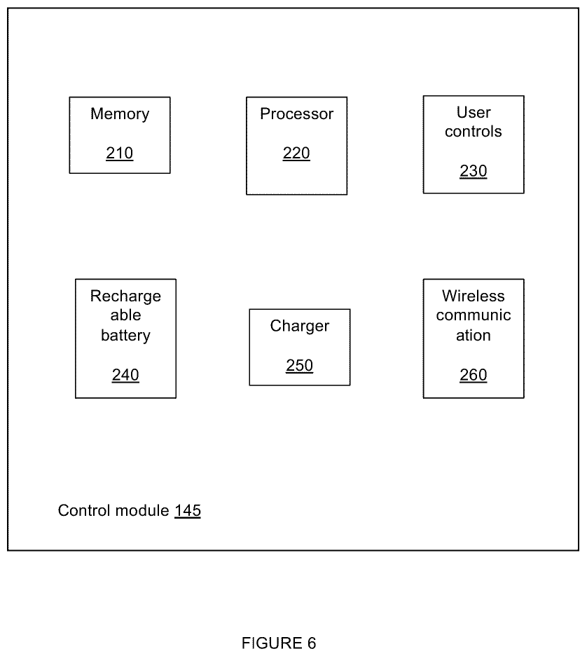

Referring now further to FIG. 6, the control module 145 comprises a memory 210 for storing instructions to be executed by a processor 220, and which when executed control the electrodes 122 of the electrode layer 120 and the heating element 135 of the heating layer 130. The control module 145 further comprises user controls 230 for controlling operation of the electrodes 122 of the electrode layer 120 and the heating element 135 of the heating layer 130. The user controls 230 may consist of manual controls, such as for example on/off button, + and − buttons, a slider, a rotary dial or any other type of manual controls for controlling intensity, or any other type of manual controls known in the art to control electrodes 122 and the heating element 135. The user controls 230 may include separate manual controls for the electrodes 122 of the electrode layer 120 and for the heating element 135 of the heating layer 130. In addition to user controls 230 for controlling the actuation and/or intensity of, the electrodes 122 and the heating element 135, the user controls 230 may further include a mode selection control for selecting an electric wave pattern for the electrodes 122 of the electrode layer 120 and/or a frequency of the wave used to control the electrodes 122. Wave patterns and wave frequency for TENS are well known in the art. The cover 147 of the electronic layer 140 provides access to the user controls of the control module 145. Alternately, the mode button may select a mode of operation for the menstrual pain management device from a plurality of preprogrammed programs for controlling the electrodes 122 of the electrode layer 120 and the heating element 135 of the heating layer 130 either concurrently or separately. The preprogrammed programs include for example: a low control for concurrently controlling the electrodes 122 of the electrode layer 120 on low intensity and the heating element 135 of the heating layer 130 on low intensity; a high control for concurrently controlling the electrodes 122 of the electrode layer 120 on high intensity and the heating element 135 of the heating layer 130 on high intensity, a sequence of controls of the electrodes 122 of the electrode layer 120 and/or of the heating element 135 of the heating layer 130, a sequence of increases of the intensity of the electrodes 122 of the electrode layer 120 and/or of the heating element 135 of the heating layer 130, a sequence of decreases of the intensity of the electrodes of the electrode layer 120 and/or of the heating element of the heating layer 130, etc.

The user controls 230 of the control module 145 may further comprise a feedback button for receiving feedback from a user of the menstrual pain management device. Upon receipt of a feedback through the feedback button, the control module 145 adapts the control of the electrodes 122 of the electrode layer 120 and the heating element 135 of the and heating layer 130.

The cover 147 of the electronic layer 140 may cover the user controls 230 while allowing the user controls 230 to be accessed over the cover 147. Each user control 230 may be represented by an embossed button, for example a molded button including a tactile icon, such as for example a “+” button for increasing intensity of the electrodes 122 or the heating element 135, a “−” for decreasing intensity of the electrodes 122 or the heating element 135, a “ [symbol] ” icon to represent actuating of the heating element 135, and any type of embossed tactile icon which allows operation of the present menstrual pain management device 100 while being worn by a user so as to permitting discrete operation. Any of the discussed user controls 230 could thus be operated through a corresponding button or actuator provided directly with a tactile icon or a regular button or actuator with a nearby tactile icon on the top cover 147.

The control module 145 further comprises a rechargeable battery 240 for powering the control module 145, the electrodes 122 of the electrode layer 120 and the heating element 135 of the heating layer 130. Of course, those skilled in the art will understand that the expression rechargeable battery 240 is meant to encompass both a single battery or a plurality of batteries.

The control module 145 further comprises a charger 250 for recharging the battery. The charger 250 may consist of a well-known in the art battery charger with an electrical connector for hooking up to electric power through an electric cord. In another embodiment, the charger 250 is a wireless charging module for wirelessly recharging the rechargeable battery. The wireless charging module may be one of an inductive wireless charging module and a capacitive wireless charging module.

The control module 145 further comprises a wireless communication module 260 for communicating with a wireless electronic device such as for example a smart phone and more particularly with an application on the wireless electronic device adapted for communication with the menstrual pain management device 100. The wireless communication module 260 may communicate using any known standards such as for example Bluetooth, Bluetooth Low-Energy, Wi-Fi, cell phone standard, or any similar or proprietary communication standard.

The wireless communication module 260 is adapted to wirelessly communicate a recharge level of the battery to the wireless electronic device. Alternatively, the control module 145 is adapted for controlling the electrodes 122 of the electrode layer 120 to generate a haptic signal indicative of a low recharge level of the rechargeable battery.

To render the menstrual pain management device 100 water resistant, a periphery of the electronic layer 140 is fused to the base layer 110 to define a protective shell around the electrode layer 120, the heating layer 130 and the control module 145.

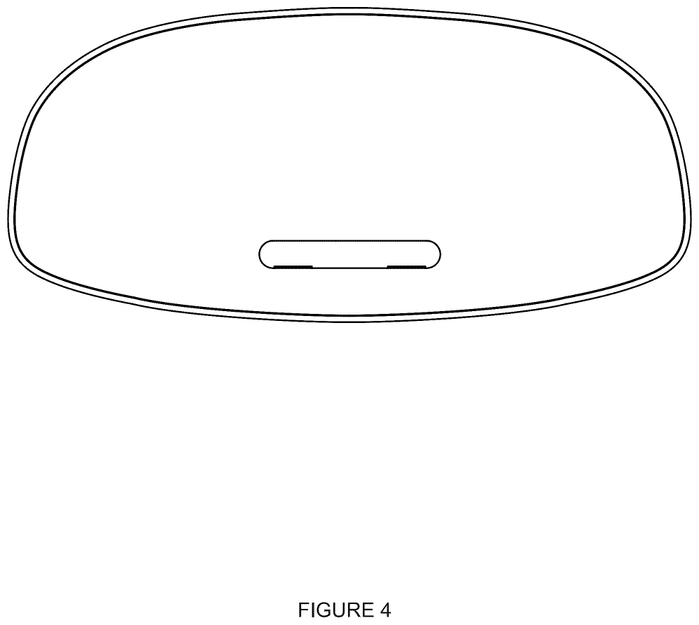

FIG. 4 illustrates an electronic base layer. For manufacturing, maintenance and repair purposes, the electronic layer 140 may be provided as an enclosed unit which is then affixed to the base layer 110, the electrode layer 120 and the heating layer 130. In this approach, only the electronic base layer shown on FIG. 4 and FIG. 5 is fused to the base layer 110, the electrode layer 120 and the heating layer 130 so as to be detachable from the electronic layer 140. Detaching the electronic layer 140 from the electrode layer 120 and the heating layer 130 facilitates cleaning of the base layer 110 without risking damage to the electronic layer 140. Furthermore, this approach allows access to the control module 145 in case a repair or maintenance is required, for example if the rechargeable battery needs replacement. Additionally, by allowing disconnection of the fused base layer 110, electrode layer 120 and heating layer 130 from the electronic layer 140, it is possible to change the fused base layer 110, electrode layer 120 and heating layer 130 to change for another type of electrodes 122 or heating element 135 without having to replace the whole menstrual pain management device 100.

Although the present disclosure has been described hereinabove by way of non-restrictive, illustrative embodiments thereof, these embodiments may be modified at will within the scope of the appended claims without departing from the spirit and nature of the present disclosure.

What is claimed is:

1. A menstrual pain management device comprising:
a base layer adapted for removable adhesion to skin;
an electrode layer, the electrode layer covering at least two areas longitudinally located along the base layer;
a heating layer;
an electronic layer, the electronic layer comprising a control module in electric connection with the electrode layer and the heating layer, the control module controlling operation of the electrode layer and the heating layer, the electronic layer covering the electrode layer and the heating layer.

2. The menstrual pain management device of claim 1, wherein the electrode layer and the heating layer are superposedly printed on one another.

3. The menstrual pain management device of claim 1, wherein:
the control module further comprises manual controls for controlling operation of the electrode layer and the heating layer; and
the electronic layer further provides access to the manual controls of the control module.

4. The menstrual pain management device of claim 3, wherein the user controls include at least one of the following: an intensity level for the electrode layer, a mode selection for selecting an electric wave pattern for the electrode layer and a frequency of the electric wave pattern.

5. The menstrual pain management device of claim 1, wherein the control module further comprises a rechargeable battery for powering the electronic module, the electrode layer and the heating layer, and an input connector for receiving a connector of an electric cable for recharging the rechargeable battery.

6. The menstrual pain management device of claim 1, wherein a periphery of the electronic layer is fused to the base layer to define a water-resistant shell around the electrode layer, the heating layer and the control module.

7. The menstrual pain management device of claim 6, wherein the electronic layer covers the controls of the control module.

8. The menstrual pain management device of claim 1, wherein the electrode layer defines a central aperture.

9. The menstrual pain management device of claim 1, wherein the control module further comprises a wireless communication module for communicating with a wireless electronic device.

10. The menstrual pain management device of claim 9, wherein the control module wirelessly communicates a recharge level of the rechargeable battery to the wireless electronic device.

11. The menstrual pain management device of claim 1, wherein the control module is adapted for controlling the electrode layer to generate a haptic signal indicative of a low recharge level of the rechargeable battery.

12. The menstrual pain management device of claim 1, wherein the control module includes a mode button for concurrently controlling the electrode layer and heating layer.

13. The menstrual pain management device of claim 12, wherein the mode button provides a low control for concurrently controlling the electrode layer on low intensity and the heating layer on low intensity.

14. The menstrual pain management device of claim 12, wherein the mode button provides a high control for concurrently controlling the electrode layer on high intensity and the heating layer on high intensity.

15. The menstrual pain management device of claim 1, wherein the base layer and the electronic layer are made of a medical grade silicon.

16. The menstrual pain management device of claim 1, wherein the electrode layer comprises a similar design of electric wire in each one of the at least two areas longitudinally located along the base layer.

17. The menstrual pain management device of claim 1, wherein the heating layer defines a belt along the base layer.

18. The menstrual pain management device of claim 1, wherein the electronic layer further comprises a rechargeable battery for powering the electronic module and a wireless charging module for wirelessly recharging the rechargeable battery.

19. The menstrual pain management device of claim 17, wherein the wireless charging module is one of an inductive wireless charging module and a capacitive wireless charging module.

20. The menstrual pain management device of claim 1, wherein the control module further comprises a feedback button for receiving feedback from a user of the menstrual pain management device, the control module adapting the control of the electrode layer and heating layer in response to the feedback received from the user.
